(19) United States
(12) Patent Application Publication
(10) Pub. No.: US 2005/0002483 A1
(43) Pub. Date: Jan. 6, 2005

(54) APPARATUS AND METHOD FOR RADIOLOGICAL IMAGE INTERPRETATION USING DIFFERENT TIME ZONES

(76) Inventor: John Richardson Wilcox JR., Athens, TX (US)

Correspondence Address: JOHN R. WILCOX, 6535 ATHENS FISH AND GAME, ATHENS, TX, 75752, US

(21) Appl. No.: 10/604,239

(22) Filed: Jul. 3, 2003

(51) Int. Cl. G06F 17/60; G21K 1/12; H05G 1/60; A61B 6/00; G01N 23/00; G06K 9/00

(52) U.S. Cl. 378/4; 382/132; 705/2

(57) ABSTRACT

A method and apparatus for high quality, timely medical interpretations of radiological images acquired in one time zone and interpreted and a different time zone. The use of a different time zone allows images acquired at night to be interpreted during regular working daylight hours. The images can include images created by conventional x-ray technology, computed radiography, magnetic resonance imaging (MRI), computed tomography (CT), ultrasound imaging, and nuclear medicine equipment. The invention includes the transmission of these images, the interpretation of these images, and the transmission of the interpretations back to the originating facility. The interpretation is performed on high-resolution workstations and the written report is created either by voice recognition software or dictation and typed transcription.

### BACKGROUND OF INVENTION

This invention relates to methods and apparatus for providing professional radiology interpretation services at locations distant from the healthcare facilities originating the studies making use of differences in time zones such that studies performed at night may be interpreted during the day thus improving the quality of the interpretation and providing continuous professional service to hospitals regardless of the time of day studies are performed.

Conventional radiology services are traditionally site-based, and therefore interpretation of imaging studies performed at night required the radiologist to also perform the interpretation at night. There are many studies that demonstrate that the normal daylight and night circadian cycle has significant effect on human work performance. The accuracy of interpreting complex diagnostic radiologic images is critically dependent upon the alertness of the individual who interprets these images. The ability to work during normal daylight hours significantly improves the alertness and accuracy of the individual performing the interpretation. Up until now hospitals have had to rely upon radiologist working at night in the hospital or from home using tele-radiology. However this results in image interpretation performed at a time when the radiologist is not at peak performance.

In view of the foregoing, it is desirable to provide a system for improving the way in which imaging studies performed at night are interpreted. Because of the availability of high speed internet connections around the world for the first time this type of work can be performed during regular daylight hours but providing interpretation to facilities at night in a different time zone. This in conjunction with voice recognition transcription the official report can be provided to the hospital in real-time thus improving accuracy, quality and efficiency of the service provided.

Other References

“The problem: shiftwork,” Costa G., Chronobiol Int 1997 March:14(2):89-98.

“Circadian rhythm, shift work and emergency medicine,” Kuhn G., Ann Emerg Med 2001 January;37(1):88-98.

“Adaptation to night shifts and synchronisation processes of night workers,” Ahasan et al., J Physiol Anthropol Appl Humman Sci 2001 July;20(4):215-26.

### SUMMARY OF INVENTION

This invention provides healthcare facilities requiring twenty four hour a day diagnostic radiologic imaging the ability to access the same standard of professional interpretation throughout the night that is routinely available during the day. It is well known that individuals who perform complex analytical and interpretive task function at a much higher level of performance if they work in phase with the normal day/night circadian cycle. Heretofore interpretation of complex medical radiologic images during the late hours of night have been done with the understanding that a more rigorous review and interpretation will be performed on the following day. This creates a situation where the standard of care that can be provided at night is not equal to that available during the day. However this invention provides a work setting and work schedule whereby an interpreting radiologist can work during normal daylight hours when interpretive skills are most accurate.

By placing the interpreting workstation site in a time zone that is sufficiently distant from the medical facility where the diagnostic images and resultant studies are acquired allows for studies performed at night to be simultaneously interpreted during the day. This is accomplished by acquiring the images in electronic digital format enabling transmission of these images over the internet to the interpreting workstation located in an appropriately distant time zone. The images at the workstation are viewed using industry standard high resolution monitors and image manipulation software. After the interpretation of the study is completed a formal report is created using industry standard computer based voice recognition software. The final electronic report is viewed on a separate monitor and then electronically signed. The computer with the voice recognition software is connected via the internet to the acquiring facility radiology information system with a virtual private network which enables acquisition of the necessary patient information and transmission of the final signed electronic report back to the acquiring site. The report is then distributed to the appropriate healthcare provider.

Thus, for the first time, complex medical diagnostic image studies acquired in the middle of the night can be interpreted simultaneously during daylight hours in a distant time zone providing the same standard of interpretation that would be available in the time zone of image acquisition on the following day. In addition the interpretation and report provided is the formal, finalized and signed report that becomes part of the official medical record.

### BRIEF DESCRIPTION OF DRAWINGS

FIG. 1 is an overview of the time relationship of the acquiring site and the workstation interpretation site.

FIG. 2 is a schematic depiction of the image and study acquisition, image transfer and reception of the completed electronic report.

FIG. 3 is a schematic depiction of how the digital image is received by the workstation, how the image is viewed and how the report is created and transmitted back to the acquiring site.

### DETAILED DESCRIPTION

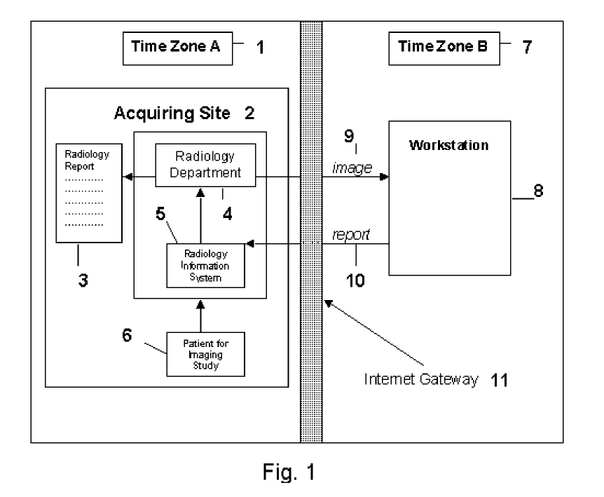

Referring to FIG. 1, in accordance with this invention, using internet based electronic transmission of radiologic images making up a radiologic study from a site acquiring the images to a workstation where the images are interpreted, in separate time zones of sufficient difference allows for the interpretation of the study to occur during daylight hours for studies performed at night. The ability of a radiologist to perform primary image interpretation of complex images during a normal circadian daylight cycle greatly improves the cognitive and analytical accuracy of the given interpretation. As depicted in FIG. 1 the time zone of the image acquisition 1 and the time zone of image interpretation 7 although separated by physical space are connected by the Internet 11. This creates a virtual state where concurrent events are at significantly different phases of the day/night cycle. The shift between Time Zone A1 and Time Zone B7 is determined by the period of the night and the length of coverage necessary for the work done in Time Zone A. Also whether to place the workstation 8 to the East or to the West of the acquiring site 2 is also determined by the period of the night and length of the night to be covered. The ideal location for the workstation 8 will vary depending on the location of the acquiring site. The following is a representative example: A busy hospital in Chicago requires continuous high quality interpretation of radiologic studies performed from 11 P. M. until 7 A. M. A workstation is placed in an office suite in Paris, France. The work period for the radiologist in Paris is from 6 A.M. until 2 P.M.

In the disclosed embodiment, the acquiring site can be any diagnostic radiology facility but would typically be a hospital requiring 24 hour a day high quality image interpretation of diagnostic radiologic studies. Such studies consisting of multiple radiological images acquired by conventional x-ray imaging, computed radiography, computed tomography (CT), magnetic resonance imaging (MRI), ultrasound imaging and nuclear medicine equipment 12.

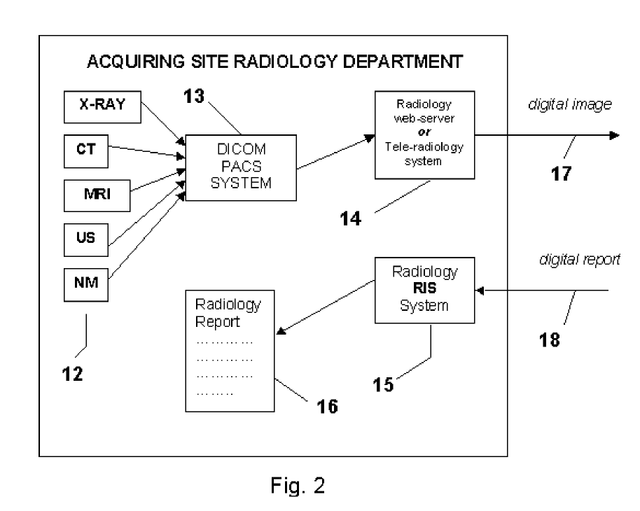

Referring to FIG. 2, the studies 12 are acquired according to Digital Imaging and Communications in Medicine (DICOM) standards and electronically transferred to a Picture Archive Computer System (PACS) 13. From the PACS 13 the images are routed by a tele-radiology system or a Radiology Web Server 14 using industry standard internet protocols 17. When the studies are transmitted by a tele-radiology system or downloaded from an image web server information identifying the patient, medical record number, date of the study and study identifying number are included with the images that make up the study. The finalized electronic report with electronic signature is returned to the acquiring site 18 over the internet with a Virtual Private Network (VPN) connection. Such a internet connection makes the transmission of information in both directions secure and complies with the Health Insurance Portability and Accountability Act of 1996 (HIPPA). The VPN connection is made with the acquiring site Radiology Information System (RIS) 15 and the voice recognition computer at the workstation site. From the RIS the report is distributed to the appropriate user or users within the acquiring site or hospital.

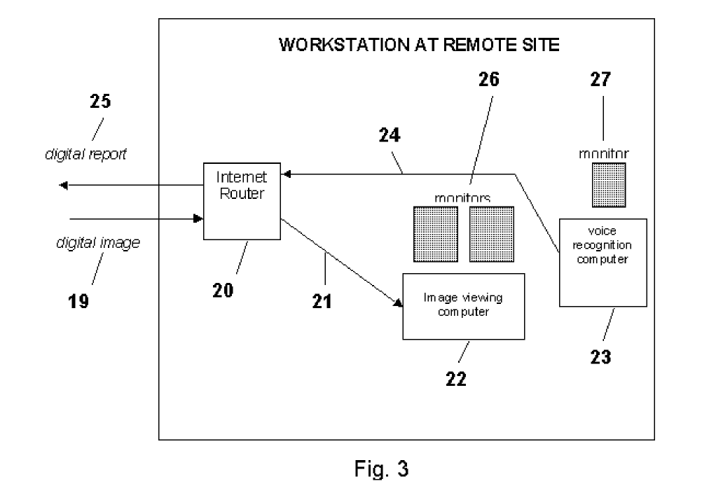

Referring to FIG. 3, the workstation site receives the images 25 transmitted over the internet at a router 20. From the router the images are downloaded into the Image Viewing Computer 22. This viewing workstation computer has from two to four high resolution flat screen monitors 26 which are used to view and interpret the images that make up the transmitted study. The computer, software program and monitors used for image viewing and interpretation may be one of several DICOM approved industry standards that are available. The official interpretation of the study performed by a radiologist is transcribed using a voice recognition computer program and computer 23 to create an electronic document. This document is viewed on a monitor and electronically signed. The computer, software program and monitor may be one of several that meet HIPPA security requirements. This computer is connected through the router 20 via the internet with the acquiring site RIS using a VPN. This connection and the voice recognition program are able to retrieve from the RIS of the acquiring site the appropriate patient identifying information and demographics of the study. The finalized electronic report is displayed on the monitor 27 of the voice recognition computer and electronically signed. The report 25 is then transmitted 24 to the acquiring site RIS 15. (The process of creating an electronic report can also be accomplished by dictation and transcription using standard word processing.)

1. A method for interpreting radiologic images:
acquired in one time zone;
interpreted in a different time zone;
the images make up a complete radiologic study;
the study is transmitted over the internet with or without compression to an image interpretation site in a different time zone.

2. The method of claim 1, further comprising: generating a report at the interpretation site; and transmitting the report to the image-acquiring site.

3. The method of claim 1, further comprising: the difference between the time zone where the study is acquired and the time zone where the study is interpreted is sufficient to enable the interpretation of studies acquire at night to be interpreted during the day.

4. The method of claim 1, wherein the medical images comprises images selected from a group comprising: radiological images, computed tomography images, ultrasound images, nuclear medicine images and magnetic resonance images.

5. The method for transmission of medical images to obtain an interpretation of the medical images, comprising a medical site with broadband internet access using a data processing system for:
performing medical imaging on a patient to obtain a plurality of medical images;
generating identifying information about a patient;
transmission of the medical images to the image interpretation site over the internet;
and at an interpretation site, using a computerized workstation for:
receiving the medical images over the internet;
displaying the medical images; generating a report relating to the medical images;
and transmitting the report to the acquiring site.

6. A method to generate a written report, comprising: a text report of the interpretation in an electronic format that can be transmitted to the acquiring site using the internet; the report may be generated using voice recognition computerized software, dictation and transcription or direct typed entry.

7. The method of claim 6, wherein the site of interpretation is able to transmit the written report in electronic form to the acquiring site and interface with the appropriate radiology information system.
